(19) United States
(12) Patent Application Publication
(10) Pub. No.: US 2014/0277032 A1
(43) Pub. Date: Sep. 18, 2014

(54) METHOD AND APPARATUS FOR MAKING ULTRASONIC IRRADIATION PLAN, AND ULTRASONIC IRRADIATION METHOD

(71) Applicant: Samsung Electronics Co., Ltd., Suwon-si (KR)

(72) Inventors: Min-su AHN, Seoul (KR); Won-chul BANG, Seongnam-si (KR)

(73) Assignee: SAMSUNG ELECTRONICS CO., LTD., Suwon-si (KR)

(21) Appl. No.: 14/202,911

(22) Filed: Mar. 10, 2014

(30) Foreign Application Priority Data
Mar. 15, 2013 (KR) 10-2013-0028238

(51) Int. Cl. A61B 19/00; A61B 17/32

(52) U.S. Cl. CPC A61B 19/50; A61B 17/320068
USPC 606/169; 703/11

(57) ABSTRACT

A method and related apparatus are provided for making an ultrasonic irradiation plan include generating a 3D organ model from an input image, generating irradiation information about a unit treatment volume based on at least one of a movement and a deformation of an organ in the 3D organ model, simulating irradiation of ultrasound for virtual treatment by using the generated irradiation information, and making an ultrasonic irradiation plan based on the simulation. Additionally, a method is provided for determining if such an ultrasonic irradiation plan is applicable, and if so carrying it out by emitting appropriate ultrasonic radiation.

### CROSS-REFERENCE TO RELATED APPLICATIONS

This application claims the benefit of Korean Patent Application No. 10-2013-0028238 filed on Mar. 15, 2013, in the Korean Intellectual Property Office, the entire disclosure of which is incorporated by reference for all purposes.

### BACKGROUND

1. Field

The following description relates to a method and an apparatuses for making an ultrasonic irradiation plan, and a method of irradiating ultrasound according to the ultrasonic irradiation plan.

2. Description of Related Art

As medical technologies have developed, local treatment of tumors has been progressed from invasive surgeries such as laparotomy to minimally-invasive surgery. Recently, non-invasive surgeries using radiation to treat tumors have been introduced. For example, such non-invasive surgeries may include a gamma knife, a cyber knife, a high intensity focused ultrasound (HIFU) knife, etc. In particular, among the listed non-invasive surgeries, the HIFU knife method that has been recently commercialized and uses focused ultrasound radiation is widely being used as a treatment that is less dangerous to a human body and environmentally-friendly.

In a HIFU treatment using a HIFU knife, a high intensity focused ultrasound is irradiated on a focus of a tumor to be treated to generate focal destruction or necrosis of the tumor's tissue, thereby removing and treating the tumor. Generally, the HIFU treatment is performed as a doctor repeats processes of irradiating ultrasound at a position of one unit treatment volume and cooling the unit treatment volume and then making a plan and performing ultrasonic irradiation and cooling for a next unit treatment volume.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Provided are a method and apparatus for making a plan of optimal ultrasonic irradiation based on movements and deformation of an organ based on a 3D organ model for a specific patient.

Also provided is a method of irradiating ultrasound, in which such an ultrasonic irradiation plan is provided to a medical doctor and the ultrasonic irradiation plan is corrected or supplemented through a diagnostic image obtained in real time, thereby enabling safe and fast surgery with respect to a dynamic organ.

Additional aspects will be set forth in part in the description which follows and, in part, will be apparent from the description, or may be learned by practice of the presented embodiments.

In an aspect, a method of making an ultrasonic irradiation plan includes generating a 3D organ model from an input image, generating irradiation information for a unit treatment volume based on at least one of a movement and a deformation of an organ in the 3D organ model, simulating ultrasound irradiation for virtual treatment by using the generated irradiation information, and making an ultrasound irradiation plan based on the simulation.

The generating of the irradiation information may include determining a position of a lesion in the organ based on the at least one of the movement and the deformation of the organ with respect to the 3D organ model, and generating irradiation information related to a transducer including at least one of a movement of the transducer and a combination of elements that are capable of irradiating in the transducer, to set a focal position of the ultrasound for virtual treatment corresponding to the determined portion of a lesion.

The generating of the irradiation information may include generating an irradiation time and an irradiation intensity of the ultrasound for virtual treatment based on the generated irradiation information related to the transducer, and the simulating of the irradiation of the ultrasound for virtual treatment may include simulating at least one of a temperature during the irradiation time and a heat accumulation amount during the irradiation time.

The input image may include a medical image obtained from a patient, and the 3D organ model is generated from the medical image.

The generating of the 3D organ model may include generating a 3D organ shape model by using a medical image, reflecting a physical characteristic for each tissue forming the organ in the 3D organ shape model, setting a target area including at least one of an obstacle, a lesion, and a protected organ in the 3D organ shape model, and generating the 3D organ model while setting a heat accumulation amount limit or a temperature limit with respect to the target area.

The simulating may include simulating a temperature or a heat treatment amount with respect to the target area in the unit treatment volume during the irradiation of the ultrasound for virtual treatment in a focal area of the unit treatment volume by using the generated irradiation information, and the making of the ultrasonic irradiation plan comprises determining whether a heat accumulation amount or a temperature with respect to the target area that is simulated exceeds the heat accumulation amount limit or the temperature limit set with respect to the target area, and if the simulated heat accumulation amount or the simulated temperature exceeds the heat accumulation amount limit or the temperature limit, generating irradiation information about a next unit treatment volume is generated, and otherwise, deleting the generated irradiation information.

The making of the ultrasonic irradiation plan may include determining whether a focal area of the unit treatment volume is necrosed by irradiation of the ultrasound for virtual treatment, in response to determining the focal area is necrosed, calculating an irradiation time of the ultrasound for virtual treatment with respect to the unit treatment volume, and in response to determining all lesions of the unit treatment volumes are not necrosed, calculating a cooling time of the unit treatment volume.

The method may provide that in the simulating of the irradiation of the ultrasound for virtual treatment a temperature or a heat accumulation amount with respect to the target area of the unit treatment volume is simulated after the cooling time is calculated.

In another general aspect, an apparatus for making an ultrasonic irradiation plan, includes a 3D organ model generator configured to generate a 3D organ model from an input image, an irradiation information generator configured to generate irradiation information for a unit treatment volume based on at least one of a movement and a deformation of an organ in the 3D organ model, a simulator configured to simulate ultrasound irradiation for virtual treatment by using the generated irradiation information, and an irradiation planner configured to make an ultrasound irradiation plan based on the simulation.

The apparatus may provide that the irradiation information generator is configured to determine a position of a lesion in the organ based on the at least one of the movement and the deformation of the organ with respect to the 3D organ model, and is configured to generate irradiation information related to a transducer including at least one of a movement of the transducer and a combination of elements that capable of irradiating in the transducer, to set a focal position of the ultrasound for virtual treatment corresponding to the determined portion of a lesion.

The apparatus may provide that the irradiation information generator is configured to generate an irradiation time and an irradiation intensity of the ultrasound for virtual treatment based on the generated irradiation information related to the transducer, and the simulator is configured to simulate at least one of a temperature during the irradiation time and a heat accumulation amount during the irradiation time.

The apparatus may provide that the input image comprises a medical image obtained from a patient, and the 3D organ model is generated from the medical image.

The apparatus may provide that the 3D organ model generator is configured to generate a 3D organ shape model by using a medical image, configured to reflect a physical characteristic for each tissue forming the organ in the 3D organ shape model, configured to set a target area including at least one of an obstacle, a lesion, and a protected organ in the 3D organ shape model, and configured to generate the 3D organ model by setting a heat accumulation amount limit or a temperature limit with respect to the target area.

The apparatus may provide that the simulator is configured to simulate a temperature or a heat treatment amount with respect to the target area in the unit treatment volume while irradiating the ultrasound for virtual treatment in a focal area of the unit treatment volume by using the generated irradiation information, and

the irradiation planner is configured to determine whether a heat accumulation amount or a temperature with respect to the target area that is simulated exceeds the heat accumulation amount limit or the temperature limit set with respect to the target area, and if the simulated heat accumulation amount or the simulated temperature with respect to the target area that is simulated exceeds the heat accumulation amount limit or the temperature limit set with respect to the protected organ, the irradiation planner is configured to generate irradiation information about a next unit treatment volume, and otherwise, the irradiation planner is configured to delete the generated irradiation information.

The apparatus may provide that the irradiation planner is configured to determine whether a focal area of the unit treatment volume is necrosed by irradiation of the ultrasound for virtual treatment, and if the focal area is necrosed, the irradiation planner is configured to calculate an irradiation time of the ultrasound for virtual treatment with respect to the unit treatment volume; and if all lesions of the unit treatment volumes are not necrosed, the irradiation planner is configure to calculate a cooling time of the unit treatment volume.

The apparatus may provide that the simulator is configured to simulate a temperature or a heat accumulation amount with respect to the target area of the unit treatment volume after the cooling time is calculated.

In another aspect, a non-transitory computer-readable storage medium stores a program for making an ultrasonic irradiation plan, the program including instructions for causing a computer to carry out the method discussed above.

In another aspect, a method of irradiating ultrasound includes generating irradiation information about a unit treatment volume based on at least one of a movement and a deformation of an organ from a 3D organ model, making an ultrasonic irradiation plan by simulating irradiation of ultrasound for virtual treatment, obtaining a diagnostic image to determine at least one of the movement and the deformation of the organ by irradiating ultrasound for diagnosis onto the unit treatment volume, comparing the 3D organ model and the obtained diagnostic image to determine whether the ultrasonic irradiation plan is applicable, and in response to determining that the ultrasonic irradiation plan is applicable, irradiating ultrasound for treatment onto the unit treatment volume.

The comparing the 3D organ model and the obtained diagnostic image to determine whether the ultrasonic irradiation plan is applicable may include predicting the at least one of a movement and a deformation of the organ from the diagnosis image, setting a position of a lesion and a focal area of the ultrasound for treatment with respect to the unit treatment volume based on a result of the prediction, calculating irradiation information of the ultrasound for treatment corresponding to the set focal area, and correcting the irradiation information of the ultrasonic irradiation plan according to the calculated irradiation information.

The method may provide that in the making of the ultrasonic irradiation plan, irradiation information about each of a plurality of unit treatment volumes including the lesion is generated, wherein if all lesions in the organ are not necrosed after the ultrasound for treatment is irradiated onto the unit treatment volume, the method further includes calculating a cooling time of the unit treatment volume and measuring a temperature or a heat accumulation amount; and setting a position of a focal area of the ultrasound for treatment with respect to a next unit treatment volume according to the ultrasonic irradiation plan.

In another aspect, an apparatus for treating a lesion with ultrasound includes an irradiation planner configured to make an ultrasonic irradiation plan based on a result of a simulated irradiation of ultrasound for virtual treatment by using irradiation information based on a 3D organ model; and a driving apparatus configured to determine whether the ultrasonic irradiation plan is applicable to a particular patient, and when the ultrasonic irradiation plan is determined to be applicable to the particular patient, irradiate ultrasound for treatment onto the patient according to the ultrasonic irradiation plan.

The ultrasonic irradiation plan may be further based on at least one of a movement and a deformation of an organ from the 3D organ model.

The determining whether the ultrasonic irradiation plan is applicable to a particular patient may be further based on comparing a diagnostic image obtained from the particular patient to the 3D organ model.

The diagnostic image may include a lesion to be treated by the ultrasonic irradiation plan.

Other features and aspects will be apparent from the following detailed description, the drawings, and the claims.

### BRIEF DESCRIPTION OF THE DRAWINGS

FIG. 1 is a diagram illustrating a configuration of an ultrasound system.

FIG. 2 illustrates a configuration of an ultrasound system of FIG. 1 for treatment.

FIGS. 3A to 3D are images illustrating the making of a plan of irradiation of ultrasound for treatment.

FIG. 4 is a flowchart illustrating a method of making an ultrasonic irradiation plan.

FIG. 5 is a flowchart illustrating another method of making an ultrasonic irradiation plan.

FIG. 6 is a flowchart illustrating in detail the method of making an ultrasonic irradiation plan of FIG. 5.

FIG. 7 is another flowchart illustrating in detail the method of making an ultrasonic irradiation plan of FIG. 5.

FIG. 8 is another flowchart illustrating in detail the method of making an ultrasonic irradiation plan of FIG. 5.

FIG. 9 is a block diagram illustrating a configuration of an ultrasonic irradiation planning apparatus of FIG. 1.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

The terms such as “first” and “second” are used herein merely to describe a variety of constituent elements, but the constituent elements are not limited by the terms. The terms are used only for the purpose of distinguishing one constituent element from another constituent element.

The terms used in the present specification are used for explaining a specific exemplary embodiment, not for limiting the present inventive concept. Thus, the expression of singularity in the present specification includes the expression of plurality unless clearly specified otherwise in context. Also, the terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

FIG. 1 is a diagram illustrating a configuration of an ultrasound system 1, according to an example embodiment. Referring to FIG. 1, the ultrasound system 1 includes a treatment ultrasound apparatus 10, a diagnostic ultrasound apparatus 20, an ultrasound data processing apparatus 30, a display apparatus 40, a driving apparatus 60, and an ultrasonic irradiation planning apparatus 90. Although FIG. 1 illustrates that the ultrasonic irradiation planning apparatus 90 is separately provided, other implementations are not limited thereto and the ultrasonic irradiation planning apparatus 90 may be provided as an element in the ultrasound data processing apparatus 30.

It is understood that the ultrasound system 1 may include other elements. For example, other embodiments may omit elements present in FIG. 1, or may replace elements present in FIG. 1 with alternative elements. Also, an external medical image 70 that is an image captured for diagnosis of a patient, such as by a medical professional, using an appropriate image acquisition apparatus (not shown) may be input to the ultrasound data processing apparatus 30 in an embodiment that will be described below.

To treat a tumor in a patient's body, the treatment ultrasound apparatus 10 of the ultrasound system 1 creates a lesion by irradiating treatment ultrasound onto a treatment portion 50 of the tumor. The diagnostic ultrasound apparatus 20 of the ultrasound system 1 irradiates diagnostic ultrasound onto a surrounding portion including the treatment portion 50 and receives a reflection wave. The surrounding portion including the treatment portion 50 will hereinafter be referred to as the observation portion. Then, the ultrasound system 1 converts the received reflection wave to an echo signal and obtains ultrasound images by processing the echo signal and diagnoses whether the treatment is complete.

The presence and scope of the lesion signifies that tissue of the treatment portion 50 locally underwent focal destruction or necrosis. The ultrasound system 1 is a system to treat the treatment portion 50 by using the treatment ultrasound apparatus 10 that irradiates, for example, the treatment ultrasound onto a part of a tumor, and monitors treatment results such as temperature of the treatment portion 50 by using the diagnostic ultrasound apparatus 20 that irradiates diagnostic ultrasound onto an observation portion. By monitoring the observation portion with the diagnostic ultrasound apparatus 20 as the treatment ultrasound apparatus 10 irradiates the treatment portion 50, the diagnostic ultrasound apparatus 20 provides information about how the treatment is progressing and how best to continue with an ongoing treatment process.

The treatment ultrasound apparatus 10 may be referred to as a treatment probe. While moving under control of the driving apparatus 60, the treatment ultrasound apparatus 10 may be directed and positioned to irradiate the treatment ultrasound onto various portions of a patient's body. Also, the treatment ultrasound apparatus 10 may be made to irradiate the treatment ultrasound onto various portions of a patient's body by reconfiguring elements of the treatment ultrasound apparatus 10 to change the position of a focus at which the treatment ultrasound is irradiated while the position of the treatment ultrasound apparatus 10 remains fixed. In other words, the treatment ultrasound apparatus 10 generates the treatment ultrasound and irradiates the treatment ultrasound onto a specific localized site including tissue of a patient.

In an embodiment, high intensity focused ultrasound (HIFU) having energy sufficient to necrose a tumor in a patient's body may be used as the treatment ultrasound. In other words, the treatment ultrasound apparatus 10 in the embodiment of FIG. 1 corresponds to an apparatus for irradiating HIFU that may be referred to as the treatment ultrasound. However, in the embodiment of FIG. 1, the treatment ultrasound apparatus 10 is not limited to ultrasound or other radiation that is referred to by the term “HIFU” and any apparatus capable of irradiating focused ultrasound or other radiation similar to HIFU may be employed as the treatment ultrasound apparatus 10 according to the embodiment of FIG. 1.

Alternatively, a method of changing the position of a focus at which the treatment ultrasound is irradiated while the position of the treatment ultrasound apparatus 10 is fixed may employ a phase array (PA) method. Such a PA method assumes that the treatment ultrasound apparatus 10 includes a plurality of elements 110, as illustrated in FIG. 2. Each of the elements 110 may individually irradiate ultrasound by receiving a signal from the driving apparatus 60, and timing for irradiating ultrasound of the elements 110 may also be differently set.

Since the elements 110 individually irradiate ultrasound, the ultrasound may be irradiated along a moving lesion while the position of the treatment ultrasound apparatus 10 is fixed. If the elements are positioned differently as in FIG. 2, and only a subset of the elements are operative at a given point in time, the treatment ultrasound apparatus 10 will irradiate different areas at different times because different elements 110 direct ultrasound towards different foci.

Thus, according to the PA method, the effect of being able to target the treatment ultrasound towards a focus area that changes over time is effectively the same as the effect of the treatment ultrasound apparatus 10 irradiating ultrasound while being physically moved to redirect the treatment ultrasound. Referring to FIG. 2, the treatment ultrasound apparatus 10 is illustrated to be circular. However, the treatment ultrasound apparatus 10 may be formed in various shapes such as a rectangle, although embodiments may include a treatment ultrasound apparatus 10 of an arbitrary shape. As discussed above, the treatment ultrasound apparatus may include a surface that is totally or partially covered by the elements 110 that emit the ultrasound.

The diagnostic ultrasound apparatus 20 may be referred to as a diagnostic probe. The diagnostic ultrasound apparatus 20 under control of the driving apparatus 60 irradiates diagnostic ultrasound toward the observation portion. The observation portion may be larger than or the same as the treatment portion 50. The observation portion is chosen so as to at least include the treatment portion 50, so that the results of the treatment may be observed. Furthermore, the observation portion may include additional areas for use in providing information when choosing how treatment is to progress. In an embodiment, the observation portion is a contiguous region. Also, the diagnostic ultrasound apparatus 20 receives reflection waves of the irradiated diagnostic ultrasound from the portion of an examinee to which the diagnostic ultrasound is irradiated. In one embodiment, the diagnostic ultrasound apparatus 20 is manufactured as a piezoelectric transducer. However, other embodiments may use alternative technologies that perform similar functionality. When ultrasound in a range of 2-18 MHz from the diagnostic ultrasound apparatus 20 is transferred to a particular portion in the patient's body, the ultrasound is partially reflected from layers between many other tissues. However, certain embodiments may operate with ultrasound that has a range that is greater or lower than this range.

In particular, the ultrasound is reflected from places where there is a change in density in the inside of a body, for example, blood cells in blood plasma, small structures in organs, etc. Because the ultrasound is reflected based on the density of the subject's body, it is possible to infer information about the structure and internal arrangement of a subject. The reflected ultrasound, that is, the reflection waves, vibrate the piezoelectric transducer of the diagnostic ultrasound apparatus 20 and the piezoelectric transducer outputs electrical pulses according to the vibration. Hence, the outputted electrical pulses are ultimately representative of the density of the regions of a subject upon which the diagnostic ultrasound is irradiated.

For example, in the embodiment of FIG. 1, an echo signal obtained by converting the reflection wave received by the diagnostic ultrasound apparatus 20 may be additionally used for monitoring a change of temperature in the observation portion. In other words, the echo signal may be used for monitoring a change of temperature in the observation portion in addition to the generation of an ultrasound diagnostic image according to techniques that are generally known. Monitoring changes of temperature in the observation portion may be useful because information about temperature changes allows embodiments to determine the effects of treatment in order to ensure that cancerous regions reach a sufficiently high temperature for necrosis and non-cancerous regions are not raised temperatures that could be detrimental.

Various techniques may be used to monitor temperature in the observation portion. For example, to monitor a change of temperature in the observation portion, temperature parameters may be extracted by comparing a reference frame indicating a frame image obtained by the diagnostic ultrasound apparatus 20 before the treatment ultrasound apparatus 10 irradiates the treatment ultrasound and a current frame indicating a frame image obtained by the diagnostic ultrasound apparatus 20 after the treatment ultrasound apparatus 10 irradiates the treatment ultrasound.

A temperature image is generated by comparing these frame images. The temperature image is derived by determining a change of temperature of the observation portion with respect to the current frame. Such a temperature image is based on information about a change of temperature between the observation portion indicated on the current frame and the observation portion indicated on the reference frame. The temperature image may be generated based on a result of the extraction of temperature parameters that define temperature information between the images.

The temperature image with respect to the current frame includes an image indicating at least one of a physical quantity that is proportional to a temperature, an image indicating a relative temperature change between the observation portion indicated on the current frame and the observation portion indicated on the reference frame, and an image indicating an absolute temperature value of the observation portion indicated on the current frame. To extract the temperature parameters, a change in backscattered energy (CBE) method, an echo-shift (ES) method, a method of calculating a change of B/A, where B/A may refer to a nonlinearity parameter, or a combination thereof may be used. Alternatively, other approaches that allow embodiments to extract the temperature parameters may also be used.

For an ultrasound treatment using treatment ultrasound such as HIFU, when HIFU arrives at a certain portion of a tumor, the temperature of a tumor portion may be instantly or almost-instantly increased to 70° C. or higher due to thermal energy of HIFU. That is, when HIFU is focused at a region of the tumor, there is so much energy that the tumor assumes a high temperature quite quickly. Theoretically, it is known that tissue destruction occurs within 110 msec at a temperature of about 60° C. As such, such a high temperature produced by the HIFU radiation leads to coagulative necrosis of tissue and blood vessels in a tumor portion. Such coagulative necrosis is desirable in a tumor, as the goal of the therapy is to eliminate or attack the tumor in this manner. However, it may be helpful to ascertain that the entire tumor has experienced coagulative necrosis, and also to avoid the occurrence of coagulative necrosis in other areas.

Accordingly, whether to continue the treatment or whether the treatment is completed may be accurately determined by accurately monitoring a change of temperature in the observation portion in real time and thus the ultrasound treatment may be efficiently performed. Additionally, monitoring temperature changes in the observation may indicate next steps for continuing the treatment as it progresses. Furthermore, even when an organ in a body moves due to, for example, breathing, a change of temperature in the observation portion may be monitored in real time by gathering and comparing images as discussed above. Thus, whether the treatment ultrasound is accurately irradiated onto the treatment portion 50, whether the treatment needs to continue, or whether the treatment is completed may be accurately determined. Furthermore, if the ultrasound is not accurately irradiated onto the treatment portion 50, it is possible to use the information about temperature changes to determine how to adjust the treatment ultrasound apparatus 10 so that the ultrasound is redirected to target the tumor more accurately or redirect the ultrasound if a portion of the tumor requires more or less ultrasound energy.

Although FIG. 1 illustrates the treatment ultrasound apparatus 10 and the diagnostic ultrasound apparatus 20 as independent devices, the present inventive concept is not limited thereto and the treatment ultrasound apparatus 10 and the diagnostic ultrasound apparatus 20 may be embodied as separate parts of a single device or as a single device that emits both diagnostic and treatment ultrasound. In other words, the treatment ultrasound apparatus 10 and the diagnostic ultrasound apparatus 20 may take on a variety of structures in different embodiments that allow them to perform their functions. Also, embodiments may use a plurality of treatment ultrasound apparatuses 10 and/or diagnostic ultrasound apparatuses 20.

Although FIG. 1 illustrates that the treatment ultrasound apparatus 10 and the diagnostic ultrasound apparatus 20 irradiate ultrasound downwardly from above a patient's body, ultrasound may be irradiated in various directions, for example, upwardly from under the patient's body. That is, other directions are possible. For example, the patient may have a tumor on one lung, and the ultrasound may be irradiated through one side of the patient's body.

The driving device 60 controls the positions of the treatment ultrasound apparatus 10 and the diagnostic ultrasound apparatus 20. that is, the driving device 60 receives information about the position of the treatment portion 50 and controls the position of the treatment ultrasound apparatus 10 so that the treatment ultrasound apparatus 10 accurately irradiates the treatment ultrasound onto the treatment portion 50. Also, the driving device 60 receives information about the position of the observation portion from the ultrasound data processing apparatus 30 and controls the position of the diagnostic ultrasound apparatus 20 so that the diagnostic ultrasound apparatus 20 may accurately irradiate the diagnostic ultrasound and receive a reflection wave. By positioning the treatment ultrasound apparatus 10 and the diagnostic ultrasound apparatus 20 as just discussed, the driving device 60 is able to ensure that these elements of the embodiments assume positions that help them accomplish their functions.

When the treatment ultrasound apparatus 10 is used in accordance with the PA method discussed above, an alternative means of focusing the ultrasound energy may be used instead of or in addition to repositioning the treatment ultrasound apparatus 10. When using the PA method, the ultrasound data processing apparatus 30 measures a displacement in which an organ moves according to a biological process, such as breathing, and calculates timing of irradiating ultrasound by each of the elements 110 forming the treatment ultrasound apparatus 10 according to the movement of the treatment portion 50 in the organ. That is, certain ones of the elements 110 forming the treatment ultrasound apparatus 10 are active at different times as the organ moves. Since the individual elements 110 direct ultrasound energy towards different absolute positions, such an approach allows the absolute position that is subject to ultrasound to vary as organs move, providing the ability to coordinate the ultrasound focus with the respiration of the patient. The ultrasound data processing apparatus 30 transmits the calculated timing information to the driving device 60. The driving device 60 transmits a command to irradiate the treatment ultrasound to each of the elements 110 forming the treatment ultrasound apparatus 10 according to the received timing information, providing utility as just discussed.

Although it is not illustrated in FIG. 1, the ultrasound system 1 may include a temperature monitor configured to measure temperature or a heat accumulation amount, a tracker configured to track a lesion or an organ to treat a movable organ, a movement anticipator configured to anticipate a movement or deformation of an organ by using a real-time 2D or 3D medical image, etc. Such additional elements may be embodied in the ultrasound data processing apparatus 30. For example, these additional elements may include specialized hardware that assists these elements in performing their tasks, as discussed above. However, it is not required that the ultrasound system 1 require specialized or discrete components for performing these tasks, and the operations performed by these optional elements may also be integrated into or performed by the other elements illustrated in FIG. 1, or by other appropriate elements that effectuate the embodiment of FIG. 1.

The ultrasonic irradiation planning apparatus 90 automatically selects the position and treatment ordering of a focal area of the treatment ultrasound apparatus 10. The ultrasonic irradiation planning apparatus 90 calculates information about at treatment plan for irradiation of the treatment ultrasound apparatus 10. For example, a treatment plan may include operations for moving the treatment ultrasound apparatus 10 to a position to necrose a lesion, selecting or combining the elements capable of irradiating ultrasound in the treatment ultrasound apparatus 10, or time or intensity to irradiate ultrasound in the selected elements. Such operations cause the treatment ultrasound apparatus 10 to operate in a manner that irradiates a changing portion of the patient with ultrasound energy in a controlled way so as to pursue medical goals while avoiding danger. In an example embodiment, the ultrasonic irradiation planning apparatus 90 makes an irradiation plan by anticipating the movement and a change in the shape of an organ in consideration of the characteristics of each patient. Thus, for the dynamic organ such as liver, kidney, pancreas, etc. that moves or has a shape change according to breathing or another biological process, a difference between the established irradiation plan and a result of surgery based on the irradiation plan may be reduced. Such reduction is possible because of techniques presented herein that model the effects of the biological process for a specific patient that lead to movement or deformation of organs. Since the changes are modeled, the ultrasonic irradiation planning apparatus 90 makes a treatment plan that compensates for the changes.

A detailed configuration of the ultrasonic irradiation planning apparatus 90 is described below with reference to FIG. 9. Referring to FIG. 9, the ultrasonic irradiation planning apparatus 90 includes a 3D organ model generator 91, an irradiation information generator 92, a simulator 93, and an irradiation planner 94.

The 3D organ model generator 91 generates a 3D organ model for each individual from an input image. The input image is a medical image of each patient. For example, the source used for an input image may include diagnostic images such as CT images, MRI image, or ultrasound images and may also be other types of 2D or 3D medical images. Also, the input image may be the external medical image 70 of FIG. 1. The 3D organ model generator 91 generates a 3D organ model shape from the input image and reflects physical features of each tissue forming an organ. For example, the physical features may include a density or an attenuation rate. The 3D organ model generator 91 includes, as part of generating a 3D organ model, setting in the 3D organ model shape a target area including an obstacle, a lesion, or a protected organ and a heat accumulation amount limit or a temperature limit for the target area. By setting such a limit, it is possible to help control the amount of heat that reaches the target area and, by so doing, protect the target area from unintended necrosis. The heat accumulation amount limit signifies a reference amount that is used to determine whether an object, for example, tissue of an organ, stops its function as heat over a certain amount is accumulated due to its characteristic and becomes necrotic. Different organs and tissues may have different heat accumulation amount limits based on factors such as their density, so it is important that the heat accumulation amount limits be chosen properly for a given target area.

Also, the target area includes a focal area where a lesion is located, a protection area around a lesion, or an area where an obstacle exists. An obstacle refers to tissue that prevents an ultrasound beam from arriving at a lesion. Tissue may act as an obstacle for various reasons. For example, when ultrasound arrives at one type of obstacle, the density of a medium through which the ultrasound propagates is sharply changed so that the ultrasound is reflected or refracted not arriving at the lesion that is a target position. The obstacle includes, for example, bones or air. The 3D organ model may be expressed in the form of a volume, a set of points, or a mesh.

The individual 3D organ model is used, in an embodiment. The shape of a generic 3D deformable organ model is deformed and matched with information about a surface of an organ obtained from CT or MRI images of a patient, information about the relative position to peripheral organs, and information about anatomical features of the inside of an organ. Such information allows such an embodiment to ensure that the generic 3D model has been adapted and customized to features of a particular patient, to improve the quality and relevance of the model in treating that particular patient. In the above process, deformation between individuals is ascertained and the generic 3D model is changed to an individual model. Thus, the individual 3D organ model representative only of the deformation of the inside of a particular individual may be generated.

Also, lesion position modeling in the individual 3D organ model adds the position of a lesion corresponding to the difference between individual patients to an individual 3D organ shape model, as a characteristic of a particular patient's individual 3D organ model. For example, the lesion may be a cyst, a region of calcification, or a tumor.

The irradiation information generator 92 generates information about irradiation with respect to a unit treatment volume in consideration of the movement or deformation of an organ in the 3D organ model generated by the 3D organ model generator 91, as discussed above. The irradiation information generator 92 determines the position of a lesion in an organ based on the movement or deformation of the organ with respect to the 3D organ model. For example, although a lesion is present in the 3D organ model in a fixed state, in consideration of a case in which the organ is moved or deformed, the position of the fixed lesion is moved and thus a new position of the lesion obtained by reflecting the movement or deformation is determined. That is, as the embodiment represents movement or deformation of the 3D organ model based on a biological process of the patient, such as respiration, it is able to use the previously derived information about the location of the lesion with respect to the organ to deduce the effects caused by the movement or deformation of the organ on the lesion to help track the position and shape of the lesion.

To set a focal position to make the lesion necrosed by irradiating ultrasound for virtual treatment at the position of the lesion according to the movement or deformation of the organ, the movement of a transducer, the selection of the elements capable of irradiating ultrasound in the transducer, or a combination of the selected elements is generated as irradiation information related to a transducer. The transducer is interpreted to have the same meaning as the treatment ultrasound apparatus 10 of FIG. 1. Although a plurality of elements may be present in examples the transducer, implementations are not limited thereto. In general, the transducer is an apparatus that produces ultrasound for treatment of lesions, as has been discussed. These various ways of changing the focal position of the ultrasound can then be used in accordance with the derived conclusions about the changing position and shape of the lesion, based on the related information about changes to the organ, to allow improved accuracy when focusing the ultrasound on the lesion.

The irradiation information generator 92 generates irradiation time and intensity to necrose a lesion by using the irradiation information related to the transducer and calculates the change in a temperature of a target area, for example, a protected organ, for the irradiation time and a heat accumulation amount for the irradiation time. By making these calculations, the irradiation information generator 92 is able to produce a sequence of instructions for the transducer that cause the transducer to emit irradiation that necroses the lesion while avoiding harm to the patient. In an embodiment, an ultrasonic irradiation plan is made by using the unit treatment volume as an irradiation unit. A unit treatment volume is an area of defined sizes that is a constituent component of the targeted lesion. An irradiation plan is established for each unit treatment volume. In an embodiment, an irradiation plan is established in which all lesions are necrosed through the irradiation plan of all unit treatment plans. Such an irradiation plan may be used to perform an actual ultrasonic irradiation.

FIGS. 3A to 3D are images illustrating the making of a plan of irradiation of ultrasound for treatment. To treat myoma uteri, the treatment portion 50 is divided into unit treatment volumes and ultrasound is irradiated many times based on individually treating each of the unit treatment volumes.

Referring to FIGS. 3A through 3C, to necrose a tumor existing in a womb and having a ball shape with a diameter of about 10 cm, the treatment portion 50 is divided into unit treatment volumes in an overall volume of a size of 20×20×40 cm³.

FIGS. 3A and 3B are a front view and a side view, respectively, and FIG. 3C illustrates focal areas of twenty six (26) unit treatment volumes. Thus, the irradiation of ultrasound according to an example embodiment performs a total of 26 irradiation processes and 25 cooling processes. As illustrated in FIG. 3D, a plan of irradiating ultrasound for all of the 26 unit treatment volumes is first made and then first irradiation and first cooling, second irradiation and second cooling, etc. are repeatedly performed with respect to the unit treatment volumes, based on the overall plan.

The simulator 93 simulates a heat accumulation amount or a temperature with respect to a target area of a unit treatment volume while irradiating ultrasound in the focal area of a unit treatment volume by using a virtual transducer based on irradiation information generated by the irradiation information generator 92. For example, the simulator 93 may receive irradiation information from the irradiation information generator 92 and then model an irradiation process in order to predict the results of the irradiation therapy and subsequently use the model as a guide for actual therapy. Also, when the focal area of the unit treatment volume is simulated to be necrosed, unless all lesions are necrosed, a cooling time is calculated and the heat accumulation amount and temperature of the target area of a next unit treatment volume is simulated.

The irradiation planner 94 determines whether the heat accumulation amount or temperature of a protected organ exceeds a simulation result of the simulator 93, for example, a heat accumulation limit or a temperature limit set to the protected organ. In other words, virtual ultrasound is irradiated according to generated irradiation time and irradiation intensity for the plan, as provided by the irradiation information generator 92. Then, only when the irradiation does not exceed the heat accumulation amount limit of an organ to be protected according to the simulator 93, irradiation information about a next unit treatment volume is checked. If the heat accumulation limit of an organ to be protected is exceeded, the previously generated irradiation information is corrected. Thus, when the organ to be protected by the virtual ultrasonic irradiation is not protected, the irradiation information about a unit treatment volume is deleted and a process of generating irradiation information again and a simulation process are repeated. Such a process uses the new information to generate a new plan that uses new parameters to attempt to necrose the lesion while avoiding damaging the surrounding tissue.

The irradiation planner 94 determines whether a focal area of a unit treatment volume is necrosed by virtual ultrasonic irradiation and, if the focal area is necrosed, an accumulated irradiation time of the virtual ultrasound with respect to the unit treatment volume is calculated. When all lesions of the unit treatment volumes are not necrosed, a cooling time for the unit treatment volume is calculated. Thus, as illustrated in FIG. 3D, the irradiation planning unit 94 makes an ultrasonic irradiation plan with respect to the irradiation and cooling of all unit treatment volumes.

The ultrasonic irradiation planning apparatus 90 according to an example embodiment may accurately and safely perform ultrasonic irradiation by reflecting in making an irradiation plan all processes of determining the position and direction of irradiation to irradiate ultrasound to a desired position, the intensity and time of irradiation to perform necrosis as large as a desired volume, and irradiation and cooling time to protect a protected organ during an ultrasound treatment. Thus, by the time the ultrasonic therapy occurs, the ultrasonic irradiation planning apparatus 90 has developed a plan, using models and simulation as discussed above, for example, that allows effective, yet safer, therapy for the patient.

FIG. 4 is a flowchart illustrating a method of making an ultrasonic irradiation plan according to an example embodiment. An ultrasonic irradiation method described with reference to FIG. 4 may be a method of irradiating an ultrasound for treatment, for example, a HIFU treatment. The method of FIG. 4 may be used in conjunction with the ultrasound system 1 of FIG. 1.

Referring to FIG. 4, an irradiation position and a focus size of ultrasound for treatment are set in operation 400, and irradiation time and intensity of ultrasound for treatment are set in operation 402. In general, operations 400 and 402 are performed using settings based on the determination or judgment of a medical doctor, or another health professional. In the example embodiment, the settings are determined by the ultrasonic irradiation planning process established by the ultrasonic irradiation planning apparatus 90, as discussed above with respect to FIGS. 3A-3D.

In operation 404, ultrasound for treatment is irradiated. The ultrasound for treatment may be irradiated for a plurality of unit treatment volumes to necrose a lesion as illustrated in FIGS. 3A through 3C, but implementations are not limited thereto and the ultrasound for treatment may be irradiation for a single treatment volume, or any appropriate group of unit treatment volumes. Additionally, while the unit treatment volumes are usually of the same shape and define a contiguous volume, embodiments may use unit treatment volumes of various shapes and define regions that are not contiguous.

In operation 406, whether normal tissue is protected is determined. If the normal tissue is not protected, the surgery is terminated. Otherwise, when it is determined that the normal tissue is protected, it is determined in operation 408 whether the focal area is necrosed. When it is determined in operation 408 that the focal area is necrosed, it is determined whether an entire lesion is necrosed in operation 410. If the entire lesion is necrosed, the surgery is terminated. Otherwise, when there remains a portion of the entire lesion that is not necrosed, a cooling time for next irradiation of ultrasound for treatment is determined in operation 412 and the process returns to operation 400. That is, the cooling time is determined and allowed to elapse before the next irradiation of ultrasound proceeds and the operations of the flowchart begin again.

FIGS. 5 through 8 are flowcharts for explaining a method of making an ultrasonic irradiation plan according to another embodiment. The ultrasonic irradiation method described with reference to FIG. 5 includes a 3D organ model generation process, a preliminary irradiation planning process, and a preliminary irradiation plan application process.

Referring to FIG. 5, a 3D organ model is generated in operation 500. The 3D organ model may reflect characteristics of an individual patient and is generated by using 2D or 3D medical images of an individual patient. A process of generating a 3D organ model is described further with reference to FIG. 6.

A 3D organ shape model is generated in operation 600. A 3D organ shape model is generated by using medical images of a patient. For example, such images may be obtained by diagnostic imaging equipment such as CT or MRI. However, other medical imaging techniques may be used. In examples, the 3D organ shape model is expressed in the form of a volume, a set of points, or a mesh.

In operation 602, physical characteristics for each tissue of an organ are reflected in the generated 3D organ shape model. The physical characteristics are physical values that are used to calculate the temperature and heat accumulation amount of irradiation of virtual ultrasound. For example, the physical characteristics may include density, attenuation rate, etc. However, the physical characteristics are not limited to these and any appropriate physical data that aids in the modeling of temperature and heat accumulation may be used.

An obstacle, a lesion, or a protected organ is set in the 3D organ shape model in operation 604. A heat accumulation amount limit or a temperature limit is set in operation 606. A 3D organ shape model for each individual is generated by setting the obstacle, the lesion, or the protected organ in the 3D organ shape model and the heat accumulation amount limit or the temperature limit for the above area. Information about the physical characteristics and the heat accumulation amount limit or the temperature limit reflected or set in operation 602 and 604 may be input by a user or a medical doctor. Alternatively, other individuals may input such information, such as other health care professionals, or the settings may be retrieved from an external source, such as a database or other information repository that contains sufficient information to control these settings. Various aspects of the use of this information have been discussed above, and will be further discussed below.

A preliminary irradiation plan is made in operation 502. The preliminary irradiation plan is made based on the individual 3D organ model generated in operation 500, and will be discussed below. As previously discussed, one method of generating the individual 3D organ model includes the operations provided in FIG. 6. It is determined in operation 504 whether a preliminary irradiation plan is applicable. If the application of a preliminary irradiation plan is not applicable, the program returns to operation 502 to make a preliminary irradiation plan. The determination of making a preliminary irradiation plan and whether the preliminary irradiation plan is applicable is described with reference to FIG. 7.

A movement or deformation of an organ is reflecting in the 3D organ model in operation 700. For example, it is determined how far or much the organ is moved or deformed compared to the generated 3D organ model, by reflecting information about a movement or deformation of an organ that moves according to breathing of a patient. The position of a lesion is identified on the 3D organ model in operation 702. When the organ is moved or deformed with respect to the 3D organ model in operation 700, the position of a lesion moves and thus the position of a lesion corresponding thereto is identified, based on the positional changes of the lesion based on the movement of the organ.

The position of a transducer is moved in operation 704. A combination of the elements in the transducer is set in operation 706. Irradiation time and intensity are calculated in operation 708. After the position of a lesion is determined at operation 704, irradiation information including the position of a transducer for irradiation at the position of a lesion, the setting of a combination of the elements in the transducer, the irradiation time, and the irradiation intensity is generated, based on operations 704, 706, and 708. The irradiation information includes information related to the transducer and irradiation information such as ultrasonic irradiation time and irradiation intensity.

The temperature or the heat accumulation amount is simulated in operation 710. When the virtual ultrasound is irradiated by using the irradiation information generated in operations 704 through 708, the temperature or heat accumulation amount in an irradiation area or a surrounding area during an irradiation period is simulated.

It is determined in operation 712, based on the results of operations 700 through 710 whether normal tissue is protected. If the normal tissue is not protected, the program returns to operation 704 and operations 704 through 710 are performed again. If the normal tissue is protected in operation 712, it is determined in operation 714 whether the focal area is necrosed. If it is determined in operation 714 that the focal area is not necrosed, the program returns to operation 700 and operations 700 through 712 are performed again.

On the other hand, if the focal area is successfully necrosed in operation 714, the irradiation time with respect to a unit treatment volume is accumulatively calculated in operation 716. Aspects of such a calculation are discussed, above. If all lesions are necrosed in operation 718, the program goes to a next process of applying a preliminary irradiation plan. Otherwise, a cooling time is calculated in operation 720. In other words, an irradiation plan with respect to the cooling time is made before an irradiation plan for a next unit treatment volume is made. However, the irradiation plan with respect to the cooling time is made after an irradiation plan for one unit treatment volume is made.

The temperature or heat accumulation amount is simulated in operation 722. A remaining area is checked in operation 724 and then operations 700 through 718 are repeated with respect to a next unit treatment volume. Thus, each treatment volume is processed and treated in sequence until a complete plan is ready.

Actual ultrasound is irradiated according to the previously made ultrasonic irradiation plan in operation 506. A process of applying the ultrasonic irradiation plan determined through operations 500 through 504 to the irradiation of actual ultrasound is described with reference to FIG. 8.

The position of a focus is set in operation 800. The position of a focus is set for irradiation of ultrasound with respect to a first unit treatment volume according to the ultrasonic irradiation plan made through operations 500 through 504. For example, the focus may be a portion of a tumor or another lesion that is designated as a starting point for treatment ultrasound energy to begin.

A real-time ultrasound image is received in operation 802. Although the real-time ultrasound image may be an image obtained through the diagnostic ultrasound apparatus 20 of FIG. 1, obtaining the image is not limited thereto and the real-time ultrasound image may be obtained through other diagnosis apparatuses. For example, the real-time ultrasound image may represent an image of a lesion and its surrounding areas in order to represent the lesion to gather information as the lesion is being treated.

The movement or deformation of an organ shape is identified in operation 804. It is determined whether the image obtained in operation 802 matches the previously generated 3D organ model or not. To this end, the movement or deformation of an organ shape is identified through a tracking system capable of tracking the movement or deformation of an organ in real time. The issue addressed at operation 804 is that the organs may be the same organs, but they may appear different on the ultrasound due to movement or deformation, such as during respiration.

When there is a movement or deformation of an individual 3D organ model in operation 806, the lesion and the position of a focus are tracked in operation 808.

The position of a transducer is moved in operation 810. A combination of the elements in the transducer is set in operation 812. The irradiation time and intensity are calculated in operation 814. To correct the irradiation information and the information related to a transducer during actual irradiation of ultrasound by reflecting the movement or deformation of a patient's organ, operations 810 through 814 are performed and then actual irradiation of ultrasound is performed. As discussed, operation 810 changes the focus of the irradiated ultrasound by redirecting the irradiated ultrasound, while operations 812 and 814 use a PA method in which the operational parameters of the transducer are changed to adjust the characteristics of the ultrasound emitted by the transducer.

The temperature or heat accumulation amount is measured in operation 814. To monitor a change in the temperature generated by the actual irradiation of ultrasound, the temperature change or the heat accumulation amount in an irradiation area where a lesion is located or a protection area including a protected organ other than the lesion is measured. A temperature monitoring system is provided to measure the temperature change or the heat acumination amount. Various techniques and approaches for modeling temperature and heat accumulation, such as certain ways of deriving temperature and heat information from ultrasound imagery, have been discussed above and may be used in this context.

It is determined in operation 818 whether normal tissue is protected. If the normal tissue is protected, the program goes to operation 820. Otherwise, the program returns to operation 800, in order to protect the normal tissue. Aspects of making this determination have been discussed above. For example, embodiments may have information about temperatures above which normal tissue will be injured, and use the information from operation 814 to assess a threat level.

It is determined in operation 820 whether the lesion in the focal area is necrosed. Again, information about the temperature of the lesion and expected temperatures of necrosis may be used here. If the lesion in the focal area is not necrosed, the program returns to operation 802 and repeats operations 802 through 818 a sufficient number of times until the lesion is necrosed.

If it is determined in operation 820 that the lesion in the focal area is necrosed, the irradiation time with respect to a unit treatment volume is accumulatively calculated, as discussed above.

If it is determined in operation 824 that all lesions are necrosed, the ultrasound treatment is terminated. Otherwise, the cooling time is calculated in operation 826 and the temperature or heat accumulation amount is measured in operation 828. The remaining area is checked in operation 830 and the program returns to operation 800, for the next round of ultrasound radiation.

The ultrasonic irradiation planning according to teachings herein may have the following merits compared to a method in which a medical doctor directly makes an ultrasonic irradiation plan and ultrasound is irradiated according to the plan.

For example, theoretically, a vast number of simulations are possible and thus various ultrasonic irradiation plans may be simulated. Various approaches can be suggested and compared, providing embodiments to optimize treatment results before an actual treatment occurs, potentially avoiding problems such as incomplete necrosis of the lesion or damage to surrounding tissue. An ultrasonic irradiation plan that is most suitable for a patient is selected based on the numerous simulations, thereby making an optimal ultrasonic irradiation plan. Additionally, having a pre-generated plan allows treatment to choose priorities for needs corresponding to a specific patient or situation.

Also, in the case of a medical doctor or other health professional directly making an ultrasonic irradiation plan without the above-described simulation environment while having ultrasound irradiated directly onto a patient's body, there may be a burden or physical danger to a patient due to potential trials and errors. However, according to the teachings herein, irradiation simulation may be automatically performed in a virtual environment to predict such a burden or danger in advance and provide a safer irradiation plan. Thus, in addition to providing a safer approach, the burden on a medical doctor or other health professional may be reduced.

Further, since a surgery time in addition to various other factors is taken into consideration when making an optimal ultrasonic irradiation plan, a total surgery time may be reduced and a burden on a patient may be reduced. For example, surgery time reduction may occur due to the ability to have a plan ready before surgery begins.

Still further, since a simulation environment is established considering not only a dynamic organ having a movement pattern but also an organ that changes its shape, a list of organs that are available for ultrasonic irradiation may be extended, because embodiments are able to adjust treatment for those organs in ways not previously available.

As described above, according to the one or more of the above embodiments, since an optimal ultrasonic irradiation plan is made based on an individual 3D organ model not only for a case of an organ having a movement but also for an organ having deformation, and is provided to a medical doctor or another health professional, the medical doctor or another health professional may more safely and quickly perform a surgery by using ultrasonic irradiation based on the plan. Moreover, lesions may be treated thoroughly, while preventing collateral damage.

The apparatus described herein may comprise a processor, a memory for storing program data to be executed by the processor, a permanent storage such as a disk drive, a communications port for handling communications with external devices, and user interface devices, including a display, keys, etc. When software modules are involved, these software modules may be stored as program instructions or computer readable code executable by the processor on a non-transitory computer-readable media such as read-only memory (ROM), random-access memory (RAM), CD-ROMs, magnetic tapes, floppy disks, and optical data storage devices. The computer readable recording media may also be distributed over network coupled computer systems so that the computer readable code is stored and executed in a distributed fashion. This media can be read by the computer, stored in the memory, and executed by the processor.

For the purposes of promoting an understanding of the principles of the teaching herein, reference has been made to the example embodiments illustrated in the drawings, and specific language has been used to describe these embodiments. However, no limitation of the scope of the embodiments is intended by this specific language, and the teachings should be construed to encompass all embodiments that would normally occur to one of ordinary skill in the art.

The teaching herein may be described in terms of functional block components and various processing steps. Such functional blocks may be realized by any number of hardware and/or software components configured to perform the specified functions. For example, an embodiment may employ various integrated circuit components, e.g., memory elements, processing elements, logic elements, look-up tables, and the like, which may carry out a variety of functions under the control of one or more microprocessors or other control devices. Similarly, where the elements an embodiment are implemented using software programming or software elements, such an embodiment may be implemented with any programming or scripting language such as C, C++, Java, assembler, or the like, with the various algorithms being implemented with any combination of data structures, objects, processes, routines or other programming elements. Functional aspects may be implemented in algorithms that execute on one or more processors. Furthermore, implementations may employ any number of conventional techniques for electronics configuration, signal processing and/or control, data processing and the like. The words “mechanism” and “element” are used broadly and are not limited to mechanical or physical embodiments, but may include software routines in conjunction with processors, etc.

The particular implementations shown and described herein are illustrative examples and are not intended to otherwise limit the scope in any way. For the sake of brevity, known electronics, control systems, software development and other functional aspects of the systems (and components of the individual operating components of the systems) may not be described for conciseness. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice unless the element is specifically described as “essential” or “critical”. It will be recognized that the terms “comprising,” “including,” and “having,” as used herein, are specifically intended to be read as open-ended terms of art.

The use of the terms “a” and “an” and “the” and similar referents in the context of describing the example embodiments (especially in the context of the following claims) are to be construed to cover both the singular and the plural. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the steps of all methods described herein can be performed in any suitable order unless otherwise indicated herein or otherwise clearly contradicted by context. The use of any and all examples, or exemplary language (e.g., “such as”) provided herein, is intended merely to better illuminate the teachings and does not pose a limitation on the scope unless otherwise claimed. Numerous modifications and adaptations will be readily apparent to those of ordinary skill in this art without departing from the spirit and scope of the invention.

The image display apparatus may be implemented as a liquid crystal display (LCD), a light-emitting diode (LED) display, a plasma display panel (PDP), a screen, a terminal, and the like. A screen may be a physical structure that includes one or more hardware components that provide the ability to render a user interface and/or receive user input. The screen can encompass any combination of display region, gesture capture region, a touch sensitive display, and/or a configurable area. The screen can be embedded in the hardware or may be an external peripheral device that may be attached and detached from the apparatus. The display may be a single-screen or a multi-screen display. A single physical screen can include multiple displays that are managed as separate logical displays permitting different content to be displayed on separate displays although part of the same physical screen.

The user interface may also be responsible for inputting and outputting input information regarding a user and an image. The interface unit may include a network module for connection to a network and a universal serial bus (USB) host module for forming a data transfer channel with a mobile storage medium, depending on a function of the ultrasound system 100. In addition, the user interface may include an input/output device such as, for example, a mouse, a keyboard, a touch screen, a monitor, a speaker, a screen, and a software module for running the input/output device.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

The methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The media may also include, alone or in combination with the software program instructions, data files, data structures, and the like. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

As a non-exhaustive illustration only, a terminal/device/unit described herein may refer to mobile devices such as, for example, a cellular phone, a smart phone, a wearable smart device (such as, for example, a ring, a watch, a pair of glasses, a bracelet, an ankle bracket, a belt, a necklace, an earring, a headband, a helmet, a device embedded in the cloths or the like), a personal computer (PC), a tablet personal computer (tablet), a phablet, a personal digital assistant (PDA), a digital camera, a portable game console, an MP3 player, a portable/personal multimedia player (PMP), a handheld e-book, an ultra mobile personal computer (UMPC), a portable lab-top PC, a global positioning system (GPS) navigation, and devices such as a high definition television (HDTV), an optical disc player, a DVD player, a Blue-ray player, a setup box, or any other device capable of wireless communication or network communication consistent with that disclosed herein. In a non-exhaustive example, the wearable device may be self-mountable on the body of the user, such as, for example, the glasses or the bracelet. In another non-exhaustive example, the wearable device may be mounted on the body of the user through an attaching device, such as, for example, attaching a smart phone or a tablet to the arm of a user using an armband, or hanging the wearable device around the neck of a user using a lanyard.

A computing system or a computer may include a microprocessor that is electrically connected to a bus, a user interface, and a memory controller, and may further include a flash memory device. The flash memory device may store N-bit data via the memory controller. The N-bit data may be data that has been processed and/or is to be processed by the microprocessor, and N may be an integer equal to or greater than 1. If the computing system or computer is a mobile device, a battery may be provided to supply power to operate the computing system or computer. It will be apparent to one of ordinary skill in the art that the computing system or computer may further include an application chipset, a camera image processor, a mobile Dynamic Random Access Memory (DRAM), and any other device known to one of ordinary skill in the art to be included in a computing system or computer. The memory controller and the flash memory device may constitute a solid-state drive or disk (SSD) that uses a non-volatile memory to store data.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of making an ultrasonic irradiation plan, comprising:
generating a 3D organ model from an input image;
generating irradiation information for a unit treatment volume based on at least one of a movement and a deformation of an organ in the 3D organ model;
simulating ultrasound irradiation for virtual treatment by using the generated irradiation information; and
making an ultrasound irradiation plan based on the simulation.

2. The method of claim 1, wherein the generating of the irradiation information comprises:
determining a position of a lesion in the organ based on the at least one of the movement and the deformation of the organ with respect to the 3D organ model; and
generating irradiation information related to a transducer including at least one of a movement of the transducer and a combination of elements that are capable of irradiating in the transducer, to set a focal position of the ultrasound for virtual treatment corresponding to the determined portion of a lesion.

3. The method of claim 2, wherein the generating of the irradiation information comprises generating an irradiation time and an irradiation intensity of the ultrasound for virtual treatment based on the generated irradiation information related to the transducer, and
the simulating of the irradiation of the ultrasound for virtual treatment comprises simulating at least one of a temperature during the irradiation time and a heat accumulation amount during the irradiation time.

4. The method of claim 1, wherein the input image comprises a medical image obtained from a patient, and the 3D organ model is generated from the medical image.

5. The method of claim 1, wherein the generating of the 3D organ model comprises:
generating a 3D organ shape model by using a medical image;
reflecting a physical characteristic for each tissue forming the organ in the 3D organ shape model;
setting a target area including at least one of an obstacle, a lesion, and a protected organ in the 3D organ shape model; and
generating the 3D organ model while setting a heat accumulation amount limit or a temperature limit with respect to the target area.

6. The method of claim 5, wherein the simulating comprises simulating a temperature or a heat treatment amount with respect to the target area in the unit treatment volume during the irradiation of the ultrasound for virtual treatment in a focal area of the unit treatment volume by using the generated irradiation information, and
the making of the ultrasonic irradiation plan comprises determining whether a heat accumulation amount or a temperature with respect to the target area that is simulated exceeds the heat accumulation amount limit or the temperature limit set with respect to the target area,
and if the simulated heat accumulation amount or the simulated temperature exceeds the heat accumulation amount limit or the temperature limit, generating irradiation information about a next unit treatment volume is generated, and otherwise, deleting the generated irradiation information.

7. The method of claim 1, wherein the making of the ultrasonic irradiation plan comprises:
determining whether a focal area of the unit treatment volume is necrosed by irradiation of the ultrasound for virtual treatment;
in response to determining the focal area is necrosed, calculating an irradiation time of the ultrasound for virtual treatment with respect to the unit treatment volume; and
in response to determining all lesions of the unit treatment volumes are not necrosed, calculating a cooling time of the unit treatment volume.

8. The method of claim 7, wherein in the simulating of the irradiation of the ultrasound for virtual treatment a temperature or a heat accumulation amount with respect to the target area of the unit treatment volume is simulated after the cooling time is calculated.

9. An apparatus for making an ultrasonic irradiation plan, comprising:
a 3D organ model generator configured to generate a 3D organ model from an input image;
an irradiation information generator configured to generate irradiation information for a unit treatment volume based on at least one of a movement and a deformation of an organ in the 3D organ model;
a simulator configured to simulate ultrasound irradiation for virtual treatment by using the generated irradiation information; and
an irradiation planner configured to make an ultrasound irradiation plan based on the simulation.

10. The apparatus of claim 9, wherein the irradiation information generator is configured to determine a position of a lesion in the organ based on the at least one of the movement and the deformation of the organ with respect to the 3D organ model, and is configured to generate irradiation information related to a transducer including at least one of a movement of the transducer and a combination of elements that capable of irradiating in the transducer, to set a focal position of the ultrasound for virtual treatment corresponding to the determined portion of a lesion.

11. The apparatus of claim 10, wherein the irradiation information generator is configured to generate an irradiation time and an irradiation intensity of the ultrasound for virtual treatment based on the generated irradiation information related to the transducer, and the simulator is configured to simulate at least one of a temperature during the irradiation time and a heat accumulation amount during the irradiation time.

12. The apparatus of claim 9, wherein the input image comprises a medical image obtained from a patient, and the 3D organ model is generated from the medical image.

13. The apparatus of claim 9, wherein the 3D organ model generator is configured to generate a 3D organ shape model by using a medical image, configured to reflect a physical characteristic for each tissue forming the organ in the 3D organ shape model, configured to set a target area including at least one of an obstacle, a lesion, and a protected organ in the 3D organ shape model, and configured to generate the 3D organ model by setting a heat accumulation amount limit or a temperature limit with respect to the target area.

14. The apparatus of claim 9, wherein the simulator is configured to simulate a temperature or a heat treatment amount with respect to the target area in the unit treatment volume while irradiating the ultrasound for virtual treatment in a focal area of the unit treatment volume by using the generated irradiation information, and
the irradiation planner is configured to determine whether a heat accumulation amount or a temperature with respect to the target area that is simulated exceeds the heat accumulation amount limit or the temperature limit set with respect to the target area,
and if the simulated heat accumulation amount or the simulated temperature with respect to the target area that is simulated exceeds the heat accumulation amount limit or the temperature limit set with respect to the protected organ, the irradiation planner is configured to generate irradiation information about a next unit treatment volume, and otherwise, the irradiation planner is configured to delete the generated irradiation information.

15. The apparatus of claim 14, wherein the irradiation planner is configured to determine whether a focal area of the unit treatment volume is necrosed by irradiation of the ultrasound for virtual treatment, and if the focal area is necrosed, the irradiation planner is configured to calculate an irradiation time of the ultrasound for virtual treatment with respect to the unit treatment volume; and if all lesions of the unit treatment volumes are not necrosed, the irradiation planner is configure to calculate a cooling time of the unit treatment volume.

16. The apparatus of claim 15, wherein the simulator is configured to simulate a temperature or a heat accumulation amount with respect to the target area of the unit treatment volume after the cooling time is calculated.

17. A non-transitory computer-readable storage medium storing a program for making an ultrasonic irradiation plan, the program comprising instructions for causing a computer to carry out the method of claim 1.

18. A method of irradiating ultrasound, comprising:
generating irradiation information about a unit treatment volume based on at least one of a movement and a deformation of an organ from a 3D organ model;
making an ultrasonic irradiation plan by simulating irradiation of ultrasound for virtual treatment;
obtaining a diagnostic image to determine at least one of the movement and the deformation of the organ by irradiating ultrasound for diagnosis onto the unit treatment volume;
comparing the 3D organ model and the obtained diagnostic image to determine whether the ultrasonic irradiation plan is applicable; and
in response to determining that the ultrasonic irradiation plan is applicable, irradiating ultrasound for treatment onto the unit treatment volume.

19. The method of claim 18, wherein the comparing the 3D organ model and the obtained diagnostic image to determine whether the ultrasonic irradiation plan is applicable comprises:
predicting the at least one of a movement and a deformation of the organ from the diagnosis image;
setting a position of a lesion and a focal area of the ultrasound for treatment with respect to the unit treatment volume based on a result of the prediction;
calculating irradiation information of the ultrasound for treatment corresponding to the set focal area; and
correcting the irradiation information of the ultrasonic irradiation plan according to the calculated irradiation information.

20. The method of claim 18, wherein, in the making of the ultrasonic irradiation plan, irradiation information about each of a plurality of unit treatment volumes including the lesion is generated, wherein
if all lesions in the organ are not necrosed after the ultrasound for treatment is irradiated onto the unit treatment volume, the method further comprises:
calculating a cooling time of the unit treatment volume and measuring a temperature or a heat accumulation amount; and
setting a position of a focal area of the ultrasound for treatment with respect to a next unit treatment volume according to the ultrasonic irradiation plan.
